(19) United States
(12) Patent Application Publication
(10) Pub. No.: US 2007/0012796 A1
(43) Pub. Date: Jan. 18, 2007

(54) Humidifying device for a humidor

(76) Inventor: Joachim Pohl, Ludenscheid (DE)

Correspondence Address: LADAS & PARRY LLP, 224 SOUTH MICHIGAN AVENUE, SUITE 1600, CHICAGO, IL, 60604, US

(21) Appl. No.: 10/572,494

(22) PCT Filed: Sep. 17, 2004
(86) PCT No.: PCT/EP04/10433

§ 371 Date: Mar. 17, 2006

(30) Foreign Application Priority Data
Sep. 17, 2003 (DE) 10342830.5

(51) Int. Cl. A24F 25/00; A61L 9/04

(52) U.S. Cl. 239/34; 239/40; 239/51

(57) ABSTRACT

The invention relates to a humidifying device for a humidor, comprising a humidity reservoir (9) from which the humidity that is discharged from the same can be directed within the interior of the humidor by means of evaporation ports (8a -8d) of the humidifying device (1). Said humidifying device (1) further comprises a fan (7), with the aid of which an air flow can be generated that allows the humidity to be conveyed into the interior of the humidor. A housing (2) of the humidifying device (1) is provided with a suction port (6) for the fan (7). According to the invention, the evaporation ports (8a-8d) are located in a lid (3) of the housing (2) of the humidifying device (1), the humidity reservoir (9) is disposed below the evaporation ports (8a-8d), and the air flow generated by the impeller (7a) of the fan (7) runs towards at least one of the evaporation ports (8a-8d) within the housing (2) of the humidifying device (1) such that the air flow is directed to the humidity reservoir (9) essentially parallel to the bottom side of the lid (3) of the housing (2) of the humidifying device (1), sweeps across the surface (9) of the humidity reservoir (9) while entraining the humidity discharged from the same, and flows out of the housing (2) through at least one of the evaporation ports (8a-8d).

The invention relates to a humidifier for a humidor, comprising a moisture reservoir, wherein the moisture escaping the moisture reservoir is led through evaporation openings of the humidifier into the interior of the humidor, wherein the humidifier comprises a fan, for generating an air flow, by which the moisture is transported into the interior of the humidor, wherein a housing of the humidifier is provided with a suction inlet for the fan.

Humidors are well-known and are used for the air-conditioned storage of cigars by regulating and controlling the moisture in the interior of the humidor by the humidifier appropriately. For that purpose, a passive humidifier is provided in the conventional, case-sized and case-shaped humidors, which is preferably secured at the cover of the humidor and comprises a moisture reservoir which successively releases a stored moisture as humidity into the interior of the humidor. In a disadvantageous way, a regulation or control of the relative humidity of the humidor only is possible in the way that the user checks the humidity in appropriate intervals of time and, according to his subjective perception, exposes more or less surface of the moisture reservoir. The “control” is therefore done exclusively by the user.

From U.S. Pat. No. 5,715,998 a humidifier with the features of the pre-characterizing part of claim 1 is known. In this humidifier it is provided that the moisture reservoir is disposed between the fan and a front side of the housing which contains outlet openings. Such a guidance of an air flow has the disadvantage that hereby the air flow is led through the moisture reservoir, which has the consequence that a high air flow resistance caused hereby is unfavourable as far as fluid-dynamics is concerned. Furthermore, a high fan power is necessary, resulting both in a high energy consumption and in a high noise disturbance.

From the DE 201 15 260 a humidifier is known, comprising a container for water or humidifying means, wherein the container comprises at least one opening capable of being closed by a shutter element. An electric motor being controlled by signals generated by a humidity sensor engages the shutter element. In this known humidifier it is provided that the shutter element can be opened or closed depending on the relative humidity inside the humidor. An active air-guidance by means of a fan is not provided.

From the U.S. Pat. No. 2,639,958 a humidifier for a humidor is known, which has two chambers, wherein the first chamber comprises an inlet opening for the fan. The fan comprises an exit opening leading to a second chamber. In the second chamber a moisture reservoir is disposed, comprising several horizontal rods, which contain absorptive material. Furthermore, the second chamber comprises a plurality of outlet openings for the moist air to escape. Such a humidifier can only be used as an integral element of an existing big humidor and is not suitable for retrofitting existing conventional humidors.

From the gastronomic sector a closet-like humidor is known, comprising a big storage shelf for several cigar-boxes and a plurality of trays to store single cigars. The bottom tray of this known big humidor is formed as a humidifying apparatus having a tub of high quality steel insertable into this tray. This humidifying apparatus comprises a motor-driven servo-unit by which the evaporation openings provided in the cover of the tub above the moisture reservoir can be opened and closed. Furthermore, the humidifying apparatus comprises a control unit, controlling the motor-driven servo-unit according to signals of two humidity and temperature sensors. The output signals of the two humidity and temperature sensors are lead to the control unit, wherein the first humidity and temperature sensor is disposed in an upper wooden cube of this humidor and the second humidity and temperature sensor is disposed in a lower portion of the humidor adjacent to the fan. The fan supports the supply of moisture. by directing an air flow over the open shutter openings of the moisture reservoir in order to achieve a faster and more intense supply of moisture. This is achieved by providing outlet openings in the cover of the steel tub for the air flow generated by the fan disposed in the steel tub. The air flow emerging essentially orthogonally out of the cover of the steel tub is deviated by an air baffle, so that it runs essentially parallel to the top of the cover of the steel tub, sweeps over the evaporation openings in the cover of the steel tub and takes up the moisture escaping from the evaporation openings. Such an air flow guidance not only has the disadvantage of increasing the size of the humidifying apparatus. It rather has the disadvantage that the air flow generated by the fan is not led directly over the moisture reservoir, but only over the outer surface of the steel tub comprising the evaporation openings.

The energy supply of the known humidifying apparatus is provided by batteries secured in a separate battery support. Providing a separate battery support also contributes to a relative large size of the known humidifying apparatus.

This known humidifying apparatus for a humidor enables an active control of humidity inside the humidor. But it has the disadvantage that, due to its extensive size, it only can be used for large, closet-like humidors (the dimensions of the known humidor are 430×540×430 mm). The application of the humidifying apparatus described above in “classic”, case-shaped and case-sized humidors is impossible due to the oversized dimensions relative to these humidors (in general the known active humidifying apparatus is larger than a classic humidor of common size).

It is therefore an object of the present invention to provide a compact humidifier for a humidor.

This object is achieved by the present invention in that the evaporation openings are provided in a cover of the housing of the humidifier, that the moisture reservoir is provided under the evaporation openings and that the air flow generated by a fan wheel of the fan is directed towards at least one of the evaporation openings within the housing of the humidifier, such that the air flow is guided to the moisture reservoir essentially parallel to the bottom side of the cover of the housing of the humidifier, sweeps over the surface of the moisture reservoir and therewith uptakes escaping moisture and leaves the housing through at least one of the evaporation openings.

The measures according to the invention provide in an advantageous way a humidifier for humidor having a compact size. The guidance of the air flow inside the housing of the humidifier according to the invention not only has the advantage that the air flow generated by the fan of the humidifier according to the invention immediately sweeps over the surface of the moisture reservoir which results in a higher efficiency. The guidance of the air flow inside the housing also has the advantage that a compact size is achievable since no more air guidance elements are necessary at the outside of the humidifier according to the invention.

The measures according to the invention in an advantageous way also provide an active humidifier which can be used in known case-sized humidors and therefore enables an active humidity control in “classic” humidors, too. The meassures according to the invention allow to provide an active humidity control in these classic humidors, in which up to now only passive humidity control was possible, too. Hereby in an advantageous way the storage quality of tobacco products stored in humidors provided with humidifiers according to the invention is improved. It is a further advantage of the invention that passive humidifiers in existing humidors simply can be replaced by humidifiers according to the invention and hence the functionality of existing humidors without an active humidity control is enhanced.

Furthermore, the humidifier according to the invention in an advantageous way can operate in a moisture mode as well as in an air-recirculating mode. At least one recirculating-air opening provided in the housing of the humidifier according to the invention in combination with a shutter mechanism renders it possible to direct the air flow generated by the fan either over the moisture reservoir and subsequently through the evaporation openings or through the recirculating-air opening.

An advantageous further development of the invention provides for a support element of the humidifier according to the invention, especially a hook and pile tape, by which it can be secured in the interior of the humidor, especially at its cover. Such a measure has the advantage that the humidifier according to the invention can easily be attached to the humidor.

Further advantageous further developments of the invention are the subject matter of the dependent claims.

Further details and advantages of the invention are disclosed in an exemplary embodiment of the invention, which is described in the following by reference to the figures. The figures show:

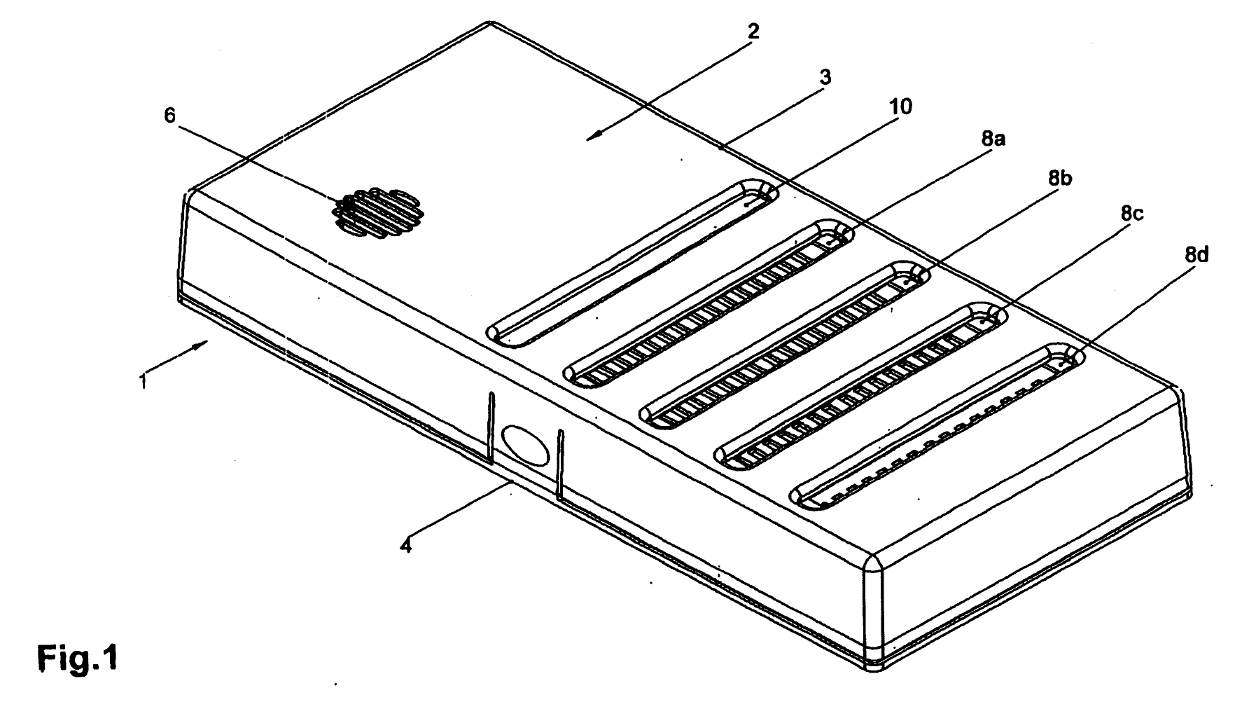

FIG. 1: an exemplary embodiment of a humidifier,

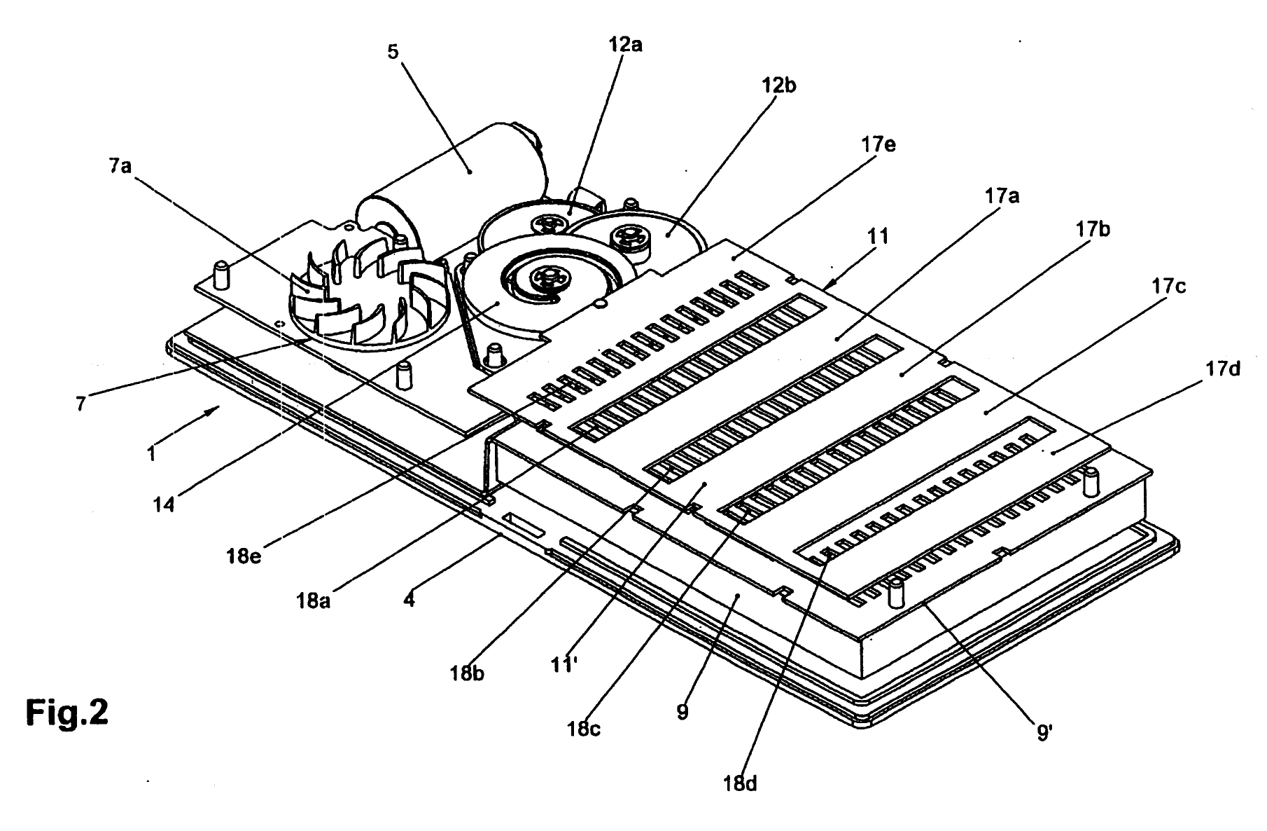

FIG. 2: the exemplary embodiment of FIG. 1 with its cover removed,

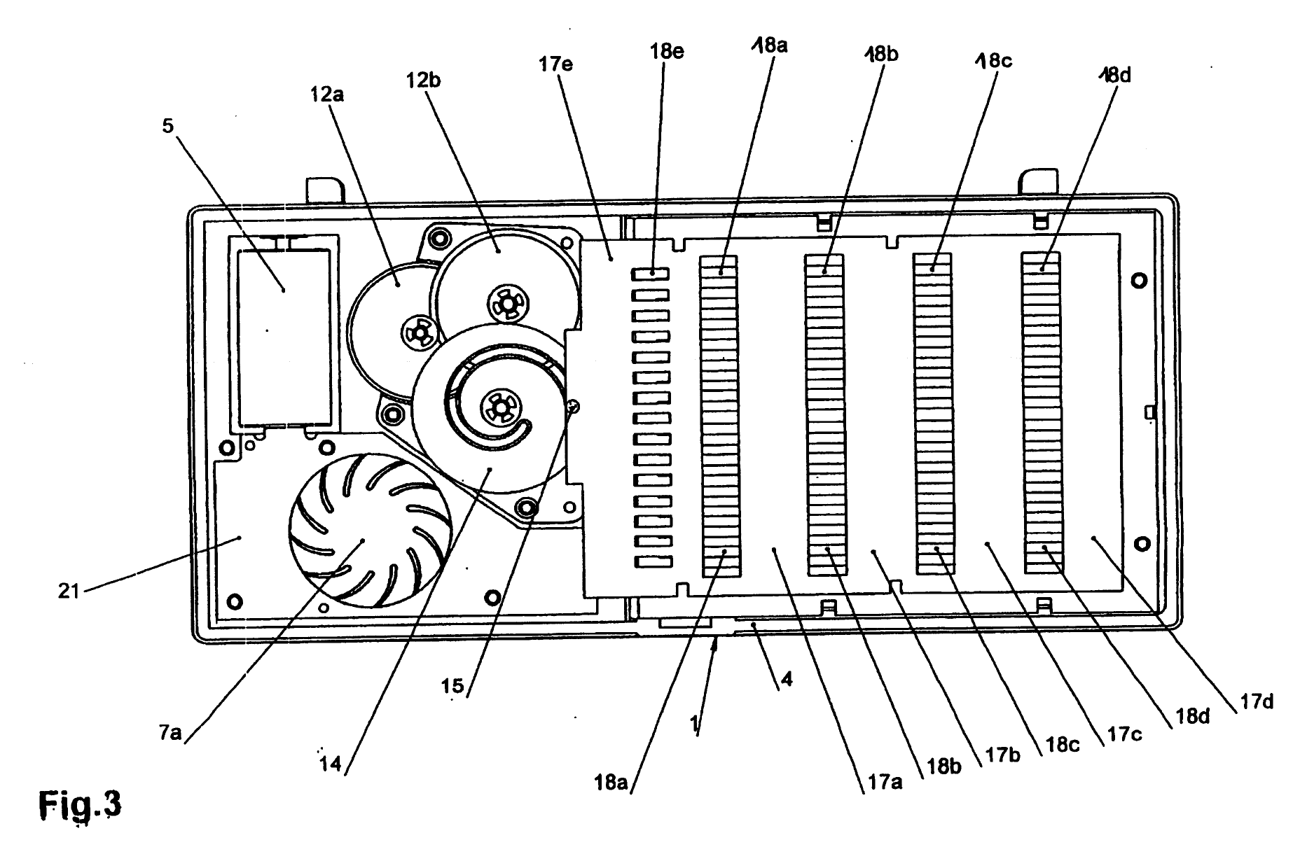

FIG. 3: a top view of the exemplary embodiment,

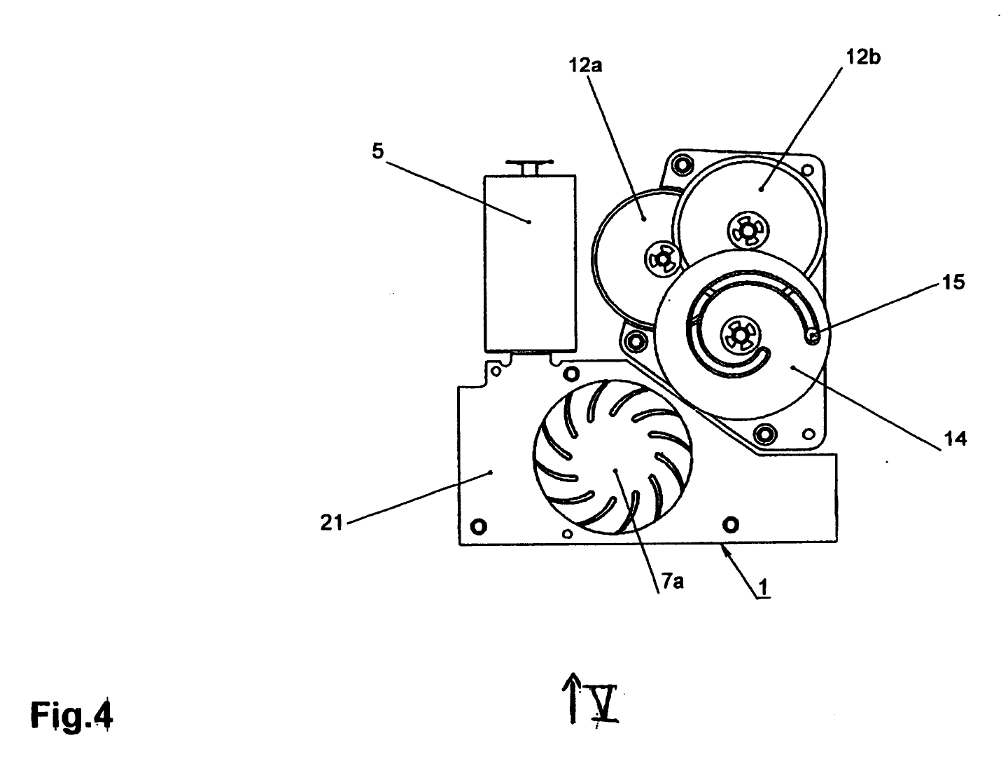

FIG. 4: a schematic top view of the several components of the exemplary embodiment,

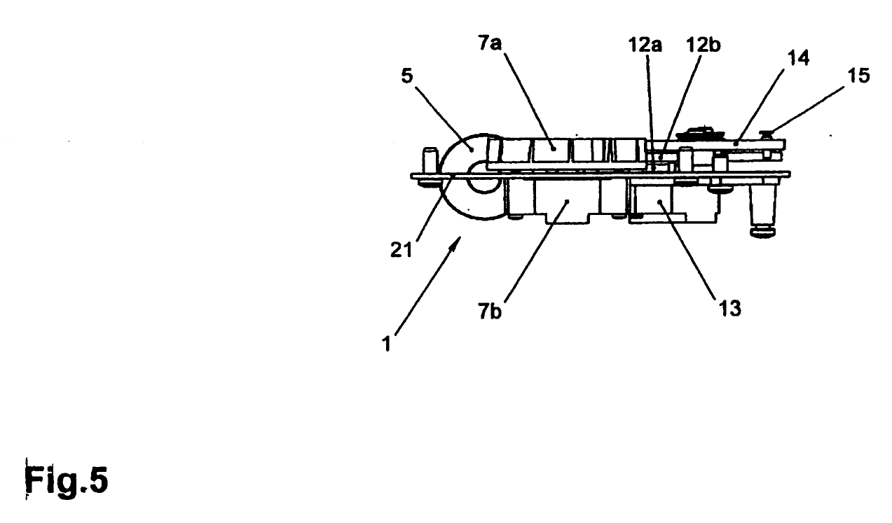

FIG. 5: a side view of the components, viewed from the direction V of FIG. 4,

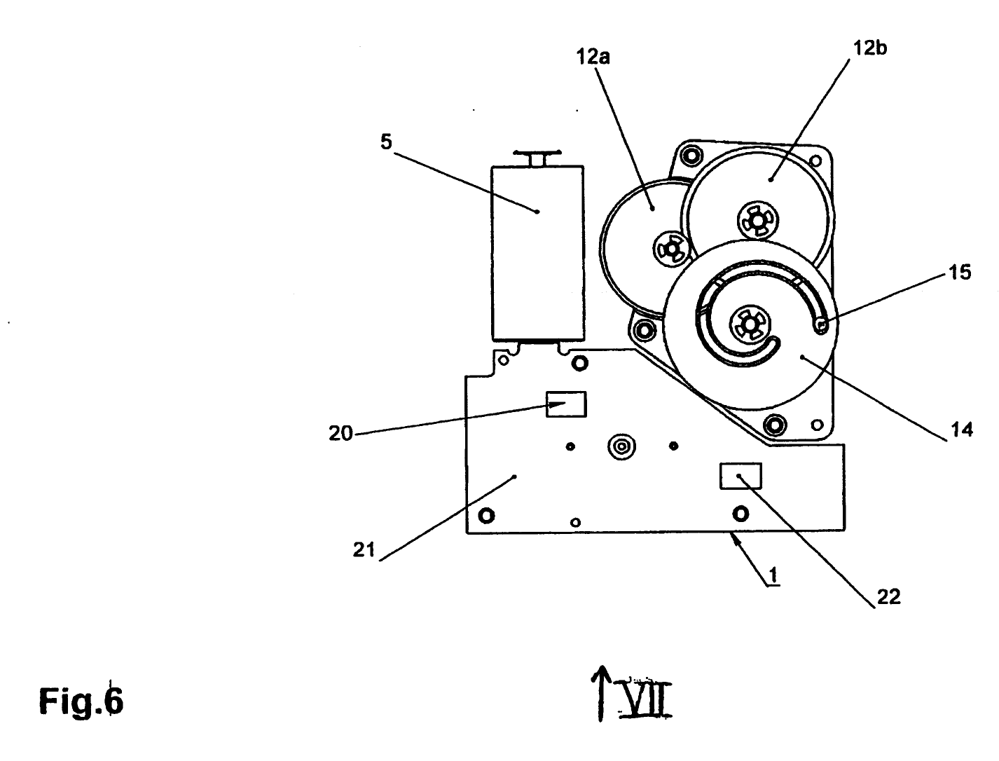

FIG. 6: a top view of the components of FIG. 4 with the fan removed, and

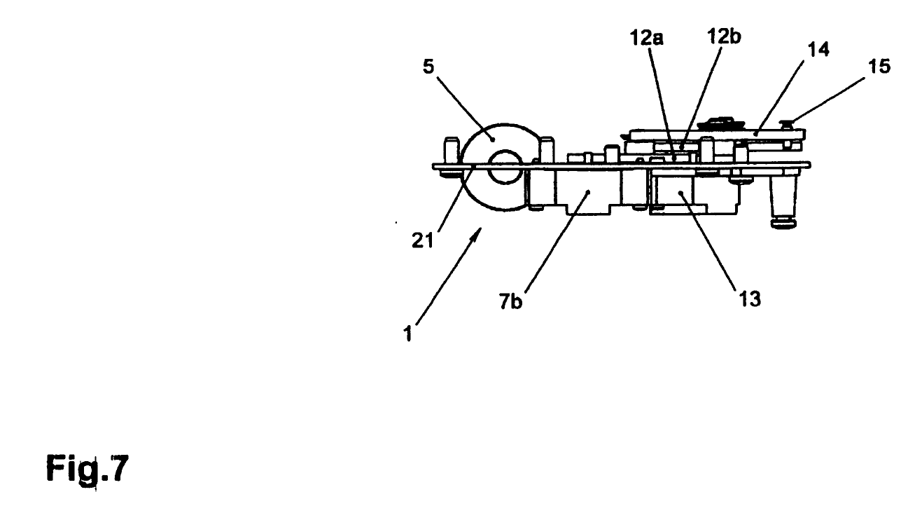

FIG. 7 a side view of the components, viewed from the direction VII of FIG. 6.

FIGS. 1 to 3 show an exemplary embodiment of a humidifier for a humidor, generally designated with reference number 1. The humidifier 1 comprises a housing 2, having a cover 3, which in the case described is detachable from a ground plate 4 of the housing 2. Such an embodiment of cover 3 composed of several parts has the advantage that the interior of the housing 2 can easily be made accessible by removing the cover 3 and therefore an easy exchange of a battery 5 secured in the housing 2 as well as the maintenance of the components of the humidifier 1 later described is possible. However, it is obvious to a person skilled in the art that the afore-mentioned removability of cover 3 of the housing 2 is not compulsory. The housing 2—although it is not preferred—rather can be composed of one single part and comprises, if required, a closeable opening for the exchange of the battery.

Preferably in the cover 3 of the housing 2, a suction inlet 6 for a fan 7 is provided, the fan wheel 7a (cf. FIGS. 2 and 3) of which is provided under the suction inlet 6. The housing 2 further comprises a plurality—in the described embodiment four—evaporation openings 8a-8d, under which a moisture reservoir 9 is provided. Furthermore it comprises at least one recirculating-air opening 10, the function of which will be described later.

The evaporation openings 8a-8d as well as the recirculating-air opening 10 can be opened and closed by a shutter mechanism 11—here provided in form of a driven slide—plate 11′—wherein this slide plate 11′ is driven by a transmission gear 12a, 12b, 14,—shown in FIGS. 5-7, which is driven by a motor 13 provided in the housing 2. The motor 13 turns a control disc 14 of the transmission gear 12a, 12b, 14. A coupling pin 15 of the shutter mechanism 11 engages the control disc 14. When the control disc 14 rotates, the coupling pin 15 displaces the slide plate 11′ provided with slots 18a-18d from its closed position, in which the evaporation openings 8a-8d are being shut by spaces 17a-17d of the slide plate 11′ provided between slots 18a-18d, to its open position, in which the slots 18a-18d of the slide plate 11′ are aligned with the evaporation openings 8a-8d. The transmission gear 12a, 12b, 14 preferably comprises a stop mechanism for limiting a displacement length of the slide plate 11′.

The air sucked in through the suction inlet 6 is led by the fan wheel 7a of the fan 7 essentially parallel to the bottom side of the cover 3 of the housing 2—i.e. inside the housing 2 of the humidifier 1—to the moisture reservoir 9, sweeps over its surface 9′, takes up moisture escaping from the moisture reservoir 9 and is then guided through the slots 18a-18d of the slide plate 11′ of the shutter mechanism 11, this slide plate 11′ of the shutter mechanism 11 being in its open-position, and exits the housing 2 through the evaporation openings 8a-8d.

The arrangement of the fan 7 in the housing 2 of the humidifier 1 in combination with the air guidance inside the housing 2 not only has the advantage that the air sucked in through the suction inlet 6 is led directly to the moisture reservoir 9 and immediately sweeps over its surface 9′, whereby a higher efficiency can be achieved, compared with the known active humidifier directing the air flow generated by the fan 7 over the evaporation openings 8a, 8d only outside of the housing and therefore only indirectly over the moisture reservoir 9. A further advantage of the described design of the humidifier 1 and the correlated guidance of the air flow generated by the fan 7 is the compact size of the described humidifier 1 for the humidor which in an advantageous way enables the described humidifier 1 also to be employed in classic, case-shaped and case-sized humidors. Therefore a retrofitting of these known humidors is possible in an easy way.

The described humidifier 1 in an advantageous way not only allows an operation in a humidifying mode, in which—as shown in FIG. 1—the evaporation openings 8a-8d are open and the moisture contained in the moisture reservoir 9 can escape, caused by the air flow generated by the fan 7, through the evaporation openings 8a-8d. But the described humidifier 1 also allows an operation in a recirculating-air mode, in which the humidity in the interior of the humidor is not increased by the moisture of the moisture reservoir 9, but the air flow generated by the fan 7 causes a circulation of the air inside the humidor. For this purpose, the motor 13 moves the shutter mechanism 11 to its closed position, in which the evaporation openings 8a-8d are shut by the spaces 17a-17d of the slide plate 11′ of the shutter mechanism 11. This effects that the recirculating-air opening 10 (cf. FIG. 1) which is shut by a space 17e in the open position of the shutter element 11, is opened so that the air flow generated by the fan 7 is able to pass through the slot 18e of the slide plate 11′ and therefore to exit the housing 2 of the humidifier 1 through the recirculating-air opening 10.

The described humidifier 1 furthermore comprises a control unit 20 (cf. FIG. 7), provided on a circuit board 21, which—for the sake of a compact size of the humidifier 1—preferably carries on its top side the fan wheel 7a of the fan 7 and on its bottom side a motor 7b driving the fan wheel 7a. The control unit 20 is fed with the signals of a—preferably digital—humidity and temperature sensor 22 which monitors the temperature and humidity in the interior of the humidor. The humidity and temperature sensor is preferably provided on the circuit board 21—preferably as an “on-chip” solution—, in order to enable a compact size of the described humidifier. The control unit 20 controls the fan 7 and the shutter mechanism 11 depending on the input signals of the humidity and temperature sensor 22 in a way that an optimal humidity is achieved in the interior of the humidor. Preferably, the humidity should be approximiately 13 g water/m³ air. The control unit 20 furthermore enables the compensation of external disturbances evoked for example by opening the cover of the humidor or by adding/taking away cigars, by a respective operation of the fan 7 and/or the shutter mechanism 11.

Preferably the control unit 20 is constructed in such a way that, due to a respective choice of its components, it is capable of directly controlling the motor 7b of the fan 7 (cf. FIG. 5) and/or the motor 13 of the shutter mechanism 11, so that in an advantageous way space-consuming amplifier stages are unnecessary.

Furthermore, in an advantageous way the battery 5 is directly connected to the circuit board 21 rendering a compact size possible. Additionally, it is provided that the battery 5 is secured in the housing 2 in a way that a misconnection of the battery 5 does not result in a destruction of the humidifier 1.

Finally it is to be mentioned, that of course the control unit 20 is capable of producing respective acoustic and/or optical alarm signals, for example if the battery 5 is no longer able to provide an appropriate energy supply, if the moisture reservoir 9 has a lack of liquid, if sensor 22 is defective, or if the cover of the humidor is opened for more than a certain period of time.

Preferably the humidifier 1 according to the invention comprises a support element by which it is attachable in the interior of the humidor, especially to its cover. In this connection the support element is preferably a hook and pile tape. This has the advantage that hereby a “smooth” attachment is possible which safely secures the humidifier 1 if the cover of the humidor is unintentionally dropped.

Concerning the embodiment of the moisture reservoir 9 it is preferred to use a liquid-containing sponge which preferably is disposed in a removable way in the moisture reservoir 9.

1. A humidifier for a humidor, comprising a moisture reservoir, wherein the moisture escaping from the moisture reservoir is led through evaporation openings of the humidifier into the interior of the humidor, wherein the humidifier comprises a fan for generating air flow by which the moisture is transported into the interior of the humidor, wherein a housing of the humidifier comprises a suction inlet for the fan, wherein the evaporation openings are provided in a cover of the housing of the humidifier, the moisture reservoir is provided under the evaporation openings, and the air flow generated by a fan wheel of the fan is led inside the housing of the humidifier towards at least one of the evaporation openings in a way that the air flow is guided essentially parallel to the bottom side of the cover of the housing of the humidifier to the moisture reservoir, sweeps over its surface and thereby takes up the moisture escaping from the moisture reservoir and exits the housing through at least one of the evaporation openings.

2. The humidifier according to claim 1, wherein the humidifier comprises at least one recirculating-air opening.

3. The humidifier according to claim 1, wherein the humidifier comprises a shutter mechanism by which at least one evaporation opening can be opened and closed.

4. The humidifier according to claim 2, wherein at least one recirculating-air opening can be opened and closed by the shutter mechanism.

5. The humidifier according to claim 2, wherein the shutting of the evaporation openings effects the opening of at least one recirculating-air opening.

6. The humidifier according to claim 3, wherein the shutter mechanism comprises a slide plate having a plurality of slots separated by spaces, that in the open-position of the shutter mechanism at least one evaporation opening is aligned with a slot, and that in the closed position of the shutter mechanism at least one evaporation opening is shut by one of said spaces of the slide plate of the shutter mechanism.

7. The humidifier according to claim 6, wherein the slide plate comprises a further slot and a further space co-operating with this slot, so that at least one recirculating-air opening can be opened and closed by said space.

8. The humidifier according to claim 3, wherein the shutter mechanism comprises a motor by which the slide plate of the shutter mechanism can be moved by means of a transmission gear.

9. The humidifier according to claim 8, wherein a control disc of the transmission gear meshes with a coupling pin of the shutter mechanism so that by a rotation of the control disc the coupling pin displaces the slide plate from its closed position to its opened position and vice versa.

10. The humidifier according to claim 8, wherein the transmission gear comprises a stop mechanism by which a displacement length of the slide plate is limited.

11. The humidifier according to claim 1, wherein the humidifier comprises a control unit by which at least one of the fan and the shutter mechanism is controllable.

12. The humidifier according to claim 11, wherein at least one motor of the fan and the motor of the shutter mechanism is directly controllable by the control unit.

13. The humidifier according to claim 1, wherein the humidifier comprises a humidity and a temperature sensor, the output signal of which is led to the control unit.

14. The humidifier according to claim 1, wherein of least one of an acoustic and an optical alarm signal can be generated by the control unit.

15. The humidifier according to claim 1, wherein the moisture reservoir comprises a sponge.

16. The humidifier according to claim 15, wherein the sponge is removably disposed in the moisture reservoir.

17. The humidifier according to claim 1, wherein the humidifier comprises a support element by which it is attachable in the interior of the humidor.

18. The humidifier according to claim 17, wherein the support element is formed as a hook and pile tape.
